(19) United States
(12) Patent Application Publication
(10) Pub. No.: US 2010/0323021 A1
(43) Pub. Date: Dec. 23, 2010

(54) ANTITUMORAL TREATMENTS

(75) Inventors: Leticia Hosta, Barcelona (ES); Mateu Pla, Barcelona (ES); Luis Javier Cruz, Barcelona (ES); Marcelo Kogan, Barcelona (ES); Fernando Albericio, Barcelona (ES)

Correspondence Address: KING & SPALDING, 1185 AVENUE OF THE AMERICAS, NEW YORK, NY, 10036-4003, US

(73) Assignee: PHARMA MAR, S.A., Madrid (ES)

(21) Appl. No.: 12/864,893

(22) PCT Filed: Jan. 30, 2009
(86) PCT No.: PCT/EP09/51080

§ 371 Date: Jul. 28, 2010

(30) Foreign Application Priority Data
Jan. 30, 2008 (EP) 08380023.5

(51) Int. Cl. A61K 9/14; C07K 7/64; A61K 38/12; A61P 35/00; B32B 5/16

(52) U.S. Cl. 424/489; 530/321; 514/21.1; 514/19.3; 428/402; 977/773; 977/810; 977/906

(57) ABSTRACT

The present invention relates to colloidal metal nanoparticles conjugated with Kahalalide F, or an analogue thereof, and their use in the treatment of cancer. The invention also relates to a method for increasing the antitumoral activity of Kahalalide F, or an analogue thereof, which comprises conjugating the Kahalalide F, on an analogue thereof, with a colloidal metal nanoparticle.

### FIELD OF THE INVENTION

The present invention relates to colloidal metal nanoparticles functionalized with Kahalalide F, or an analogue thereof, and their use in the treatment of cancer. The invention also relates to a method for increasing the cytotoxic effects of Kahalalide F, or an analogue thereof, by conjugation with a colloidal metal nanoparticle.

### BACKGROUND OF THE INVENTION

Cancer develops when cells in a part of the body begin to grow out of control. Although there are many kinds of cancer, they all start because of out-of-control growth of abnormal cells. Cancer cells can invade nearby tissues and can spread through the bloodstream and lymphatic system to other parts of the body. There are several main types of cancer. Carcinoma is cancer that begins in the skin or in tissues that line or cover internal organs. Epithelial cells, which cover internal and external surfaces of the body, including organs and lining of vessels, may give rise to a carcinoma. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system.

In addition, cancer is invasive and tends to metastasise to new sites. It spreads directly into surrounding tissues and also may be disseminated through the lymphatic and circulatory systems.

Many treatments are available for cancer, including surgery and radiation for localised disease, and chemotherapy. However, the efficacy of available treatments for many cancer types is limited, and new, improved forms of treatment showing clinical benefit are needed. This is especially true for those subjects presenting with advanced and/or metastatic disease and for subjects relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment or ultimately die from progressive metastatic disease. Thus, the ongoing commitment to the design and discovery of new anticancer agents is critically important.

Chemotherapy, in its classic form, has been focused primarily on killing rapidly proliferating cancer cells by targeting general cellular metabolic processes, including DNA, RNA, and protein biosynthesis. Chemotherapy drugs are divided into several groups based on how they affect specific chemical substances within cancer cells, which cellular activities or processes the drug interferes with, and which specific phases of the cell cycle the drug affects. The most commonly used types of chemotherapy drugs include: DNA-alkylating drugs (such as cyclophosphamide, ifosfamide, cisplatin, carboplatin, dacarbazine), antimetabolites (such as 5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase I and II inhibitors (such as topotecan, irinotecan, etoposide, teniposide), and hormone therapy (such as tamoxifen, flutamide).

The ideal antitumor drug would kill cancer cells selectively, with a wide index relative to its toxicity towards non-cancer cells and it would also retain its efficacy against cancer cells, even after prolonged exposure to the drug. Unfortunately, none of the current chemotherapies with these agents posses an ideal profile.

It has long been a goal to find the magic bullet that would track to the site of need and deliver an antitumoral response without undue side effects. By designing a particle delivery system capable of sequestering a cancer drug solely within a tumor may also reduce the accumulation of the drug in healthy organs. Consequently, these delivery systems may increase the relative efficacy and/or safety of a cancer therapy, and thus serve to increase the drug's therapeutic index.

Nanotechnology offers tremendous potential for medical diagnosis and therapy. In this sense, various types of nanoparticles have been explored for biomedical applications (Alivisatos P. Nat. Biotechnol. 2004, 22, 47-52; Kim J. et al. Angew. Chem. Int. Ed. 2006, 45, 7754-7758), and have been widely employed in biological systems.

Attractive for their size, stability and biocompatibility, gold nanoparticles have been used in a number of biomedical applications. The ability to functionalize the surface of gold with organic molecules allows for the preparation of nanoparticles which can specifically interact with any physiological system. One of the most interesting applications of gold particles in biomedicine is the use of surface modified gold nanoparticles as vehicles for drug delivery.

Colloidal gold nanoparticles represent a relatively novel technology in the field of particle-based tumor-targeted drug delivery. It has been reported the use of functionalized gold nanoparticles for the targeted delivery of the potent yet highly toxic anticancer protein, tumor necrosis factor (TNF), to a solid tumor. (Paciotti G F and Myer L. Drug Delivery, 2004, 11, 169-183). In vivo, this nanodrug actively targets and sequesters recombinant TNF in solid tumors.

More recently, there have also been described gold nanoparticles functionalized with other antitumoral drugs such as Paclitaxel (Gibson J. et al. J. Am. Chem. Soc. 2007, 129(37), 11653-61) and Metotrexate (Chen Y H et al. Mol. Pharm. 2007, 4(5), 713-22). Specifically, Paclitaxel was first attached to a hexaethylene glycol linker followed by coupling of the resulting linear analogue to phenol-terminated gold nanocrystals having a size particle of 2 nm. On the other hand, Metotrexate gold nanoparticles were prepared by direct binding of the drug onto gold nanoparticles having a particle size of 13 nm via the carboxyl groups present in Metotrexate molecule.

Tumour-targeting drug delivery vectors are now approaching “true” nanometre size, which allows them to arrive in close proximity to several biological targets (Paciotti G F et al. Drug Development Research, 2006, 67, 47-54). In this context, Tkachenko et al. have disclosed nuclear targeting by gold nanoparticles modified with nuclear localization peptides. Accordingly, gold particles were modified with a shell of bovine serum albumin (BSA) and conjugated to various cellular targeting peptides (Tkachenko A G et al. Bioconjugate Chem. 2004, 15, 482-490; Tkachenko A G et al. J. Am. Chem. Soc. 2003, 125, 4700-4701).

Cellular delivery involving the transfer of various drugs and bioactive molecules (e.g. peptides, proteins and DNA) through the cell membrane into the cytoplasm has attracted great attention because the general administration of drugs and biomolecules suffers from inefficiency and enzymatic degradation, among other problems. Hence, there is a pressing need to develop safe and efficient transport vehicles to deliver drugs into cells (Xua Z P et al. Chemical Engineering Science, 2006, 61(3), 1027-1040).

Natural products and their derivatives have traditionally been a common source of drugs. Cytotoxic peptides are synthesised by a large number of plants and animals. One class of natural products are kahalalide compounds which are cyclic depsipeptides originally isolated from a Hawaiian herbivorous marine species of mollusk, Elysia rufescens, and its diet, the green alga Briopsis sp. Kahalalides A-G were described by Hamann et al. (J. Am. Chem. Soc. 1993, 115, 5825-5826 and J. Org. Chem. 1996, 61, 6594-6600) and many of them show activity against cancer and AIDS-related opportunistic infections. Some other natural kahalalide compounds have been also disclosed such as Kahalalide H and J by Scheuer et al. (J. Nat. Prod. 1997, 60, 562-567), Kahalalide 0 by Scheuer et al. (J. Nat. Prod. 2000, 63(1), 152-154), and Kahalalide K by Kan et al. (J. Nat. Prod. 1999, 62(8), 1169-1172).

Of the kahalalide compounds, Kahalalide F (KF) and analogues thereof are the most promising because of their antitumoral activities. The structure of these compounds is complex, comprising six amino acids as a cyclic part, and an exocyclic chain of seven amino acids with a terminal aliphatic/fatty acid group. Specifically, Kahalalide F has the following structure:

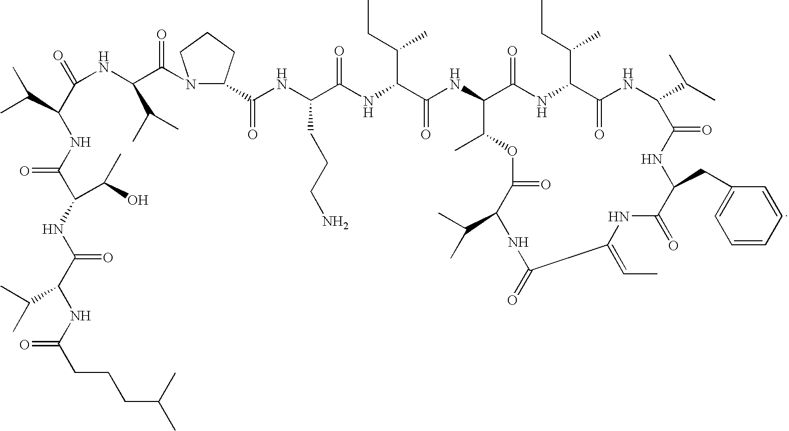

EP 610.078 reports that early preclinical in vitro screening studies identified micromolar activity of Kahalalide F against mouse leukemia (P388) and two human solid tumors: non-small cell lung (A549) and colon (HT-29). Subsequent studies identified that Kahalalide F displayed a selective in vitro and in vivo cytotoxicity profile in androgen-dependent prostate cancer and other solid tumors such as those of breast, colon, non-small-cell-lung (NSCL), and ovary, with lack of complete cross-resistance with conventional anticancer agents. In contrast, non-tumour cell lines are 5 to 40 times less sensitive to Kahalalide F (Medina L A et al. Proc. Am. Ass. Cancer Res. 2001, 42, abstr. 1139; Faircloth G et al. Proc. Am. Ass. Cancer Res. 2001, 19, abstr. 1140; Garcia-Rocha M et al. Cancer Lett. 1996, 99(1), 43-50; Suarez Y et al. Mol. Cancer. Ther. 2003, 2(9), 863-872; Sewell J M et al. Eur. J. Cancer, 2005, 41, 1637-1644).

Additionally, it has been found that soon after exposure to Kahalalide F, cells initiate a death process included marked swelling and a series of profound morphological alterations that affect many cytoplasm organelles and the plasma membrane. These features are typical of the process named oncosis, a term describing the progression of cellular events leading to necrotic cell death. The cellular architecture is greatly affected as early as 1-3 hours after Kahalalide F treatment, and the integrity of crucial organelles such as mitochondria, ER, or lysosomes is severely compromised. In contrast, the nuclear structure is preserved, and no drastic alteration of chromatin or DNA degradation is detected (Suarez Y et al. Mol. Cancer. Ther. 2003, 2, 863-872).

Kahalalide F primary mechanism of action has not been identified yet. However, it was found that Kahalalide F is an NCI-COMPARE negative compound that induces sub G1 cell-cycle arrest and cytotoxicity independently of MDR, Her2, P53, and blc-2 (Janmaat et al. Proceedings of the 2nd International Symposium on Signal Transduction Modulators in Cancer Therapy: 23-25 October, Amsterdam 2003: 60 (Abst. B02)). The COMPARE analysis in a panel of 60 human cancer cell lines genetically and molecularly characterized for cell proliferation pathways has included Kahalalide F in the list of new chemical entities that interact with the Erb/Her-neu pathway (Wosikowsky et al. J. Natl. Cancer Inst. 1997, 89, 1505-1515). Sensitivity to Kahalalide F significantly correlated with baseline expression levels of ErbB3 (HER3), but not of other ErbB receptors, in a panel of established cell lines from different origins. Furthermore, the downstream P13K/Akt pathway coupled to ErbB3 receptor is also affected by Kahalalide F treatment. Kahalalide F decreases phosphorylated Akt levels and this reduction is associated with cytotoxicity in Kahalalide F-sensitive cell lines (Janmaat et al. Mol Pharmacol 2005, 68, 502-510).

Among Kahalalide F analogues, 4-methylhexanoic analogue is of particular interest, especially its (4S)-methylhexanoic analogue (PMO2734), because of its improved efficacy shown in in vivo cancer models with respect to those activities observed with Kahalalide F. PMO2734 has demonstrated in vitro antitumor activity against a broad spectrum of tumor types such as leukemia, melanoma, breast, colon, ovary, pancreas, lung, and prostate, and has shown significant in vivo activity in xenografted murine models using human tumor cell types such as breast, prostate, and melanoma. This compound is the subject of WO 2004/035613 and has the following structure:

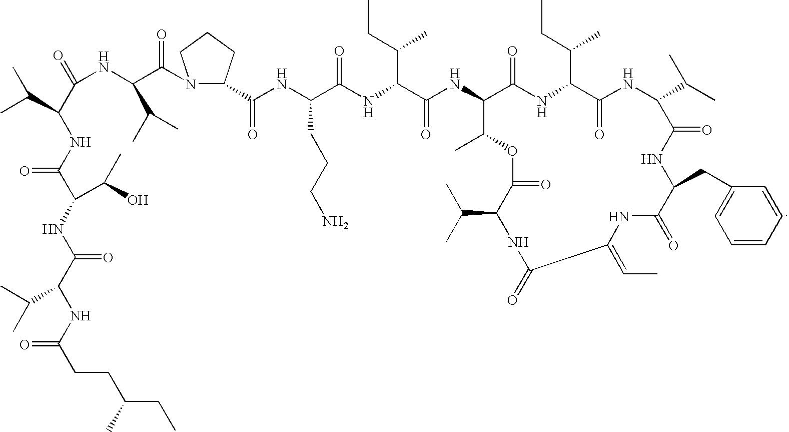

More information on Kahalalide F and analogues thereof, their uses, formulations and synthesis can be found in the patent applications EP 610.078, WO 2004/035613, WO 01/58934, WO 2005/023846, WO 2004/075910, WO 03/033012, WO 02/36145, WO 2005/103072, and U.S. 60/981,431. We incorporate by specific reference the content of each of these applications.

Since cancer is a leading cause of death in animals and humans, several efforts have been and are still being undertaken in order to obtain an antitumor therapy active and safe to be administered to patients suffering from a cancer. The problem to be solved by the present invention is to provide antitumor therapies that are useful in the treatment of cancer.

### SUMMARY OF THE INVENTION

We have established that functionalized nanoparticles obtained by conjugation of Kahalalide F or an analogue thereof with a colloidal metal nanoparticle are potentially useful in the treatment of cancer. Moreover, it has been established that Kahalalide F, or an analogue thereof, conjugated colloidal metal nanoparticles show an improved cytotoxic activity when compared with the activity of the single peptide.

In accordance with one aspect of this invention, we provide a colloidal metal nanoparticle conjugated with Kahalalide F or an analogue thereof.

In another aspect, the invention relates to a colloidal metal nanoparticle conjugated with Kahalalide F, or an analogue thereof, for use as a medicament, in particular for use as a medicament for treating cancer.

In another aspect, the invention also relates to the use of a colloidal metal nanoparticle conjugated with Kahalalide F, or an analogue thereof, for the manufacture of a medicament for the treatment of cancer.

In another aspect, the invention relates to a pharmaceutical composition comprising a colloidal metal nanoparticle conjugated with Kahalalide F, or an analogue thereof, and a pharmaceutically acceptable vehicle.

In a related aspect, the invention refers to the use of a colloidal metal nanoparticle conjugated with Kahalalide F, or an analogue thereof, in combination with another drug to provide a combination therapy for the treatment of cancer.

In another aspect, the invention relates to a method for increasing the antitumoral activity of Kahalalide F, or an analogue thereof, which comprises conjugating the Kahalalide F, or the analogue thereof, with a colloidal metal nanoparticle.

In a further aspect, the invention relates to a colloidal metal nanoparticle conjugated with Kahalalide F, or an analogue thereof, which is further conjugated to an additional agent. Furthermore, it relates the use of said conjugated colloidal metal nanoparticle for the intracellularly delivering of said additional agent.

In another aspect, the invention relates to a method of treating cancer comprising administering to a patient in need of such treatment a therapeutically effective amount of a colloidal metal nanoparticle conjugated with Kahalalide F or an analogue thereof.

In yet another aspect, the invention relates to a method for obtaining a colloidal metal nanoparticle conjugated with Kahalalide F, or an analogue thereof, comprising the following steps:
(i) obtaining colloidal metal nanoparticles by reduction of a solution of a metal salt;
(ii) mixing a solution of Kahalalide F or an analogue thereof with the colloidal metal nanoparticle solution obtained in step i) for a sufficient period of time to form conjugated nanoparticles, wherein the Kahalalide F, or an analogue thereof, is in excess with respect to the colloidal metal nanoparticle;
(iii) optionally, admixing the conjugated nanoparticles obtained in step ii), with an additional agent to form a reaction mixture and incubating the reaction mixture for a sufficient period of time to allow the conjugated nanoparticles to bind said additional agent; and
(iv) isolating the conjugated colloidal metal nanoparticles.

### BRIEF DESCRIPTION OF THE FIGURES

FIG. 1. TEM images of 20 nm gold nanoparticle solution (FIG. 1A) and of 40 nm gold nanoparticle solution (FIG. 1B).

FIG. 2. UV-vis spectra of 20 nm and 40 nm sized gold nanoparticles and their respective conjugates; a shift in the maximum with regards to the unconjugated gold nanoparticles is representative of the conjugation. (a) 20 nm AuNPs unconjugated and conjugated to P1 and to P2 and (b) 40 nm AuNPs unconjugated and conjugated to P1 and to P2.

FIG. 3. High-resolution TEM micrographs (HRTEM) of uncoated gold nanoparticles (FIG. 3A) and P1 coated gold nanoparticles (FIG. 3B). The presence of the peptide was detected upon uranyl acetate staining, shown as a layer around the nanoparticle core in FIG. 3B.

FIG. 4. (a) EELS spectrum obtained on the surface of a 20 nm non-functionalised gold nanoparticle; (b) Detail of the Au O_{2,3} ELNES spectrum of (a); (c) Detail of the S L_{2,3} edge of (a); (d) EELS spectrum obtained on the surface of a 20 nm P1-conjugated gold nanoparticle; (e) Detail of the Au O_{2,3} ELNES spectrum of (d); (f) Detail of the S L_{2,3} edge of (d).

FIG. 5. XPS S2p region spectra of gold nanoparticles on (a) PMMA, (b) P1-functionalised gold surfaces, and (c) P1-conjugated gold nanoparticles on a PMMA surface. The spectra have been normalised.

FIG. 6. Anti-proliferation results after incubation of HeLa cells for 24 h with (a) P1- and P2-conjugated 20 nm gold nanoparticles and (b) P1- and P2-conjugated 40 nm gold nanoparticles.

FIG. 7. Confocal microscopy images showing the localisation of gold nanoparticles and their conjugates in HeLa cells. Membranes were stained with a fluorescence marker (WGA), and nuclei with a DNA marker (Hoechst).

FIG. 8. TEM images of HeLa cells incubated with (a) 20 nm unconjugated nanoparticles, (b) 20 nm P1-conjugated nanoparticles, and (c) 40 nm P1-conjugated nanoparticles. The arrows indicate the presence of the AuNPs inside lysosome-like structures. Abbreviations: NU (nucleus), RER (rough endoplasmic reticulum), GA (Golgi apparatus).

### DETAILED DESCRIPTION

The authors of the present invention have established that colloidal metal nanoparticles can be functionalized by conjugation with Kahalalide F and analogues thereof. Moreover, it has surprisingly been found that the resulting functionalized nanoparticles show an improved antitumoral activity when compared with the activity of the compounds administered alone.

In order to study the feasibility and biological properties of colloidal metal nanoparticles conjugated with Kahalalide F or an analogue thereof, synthetic epimer analogues of Kahalalide F (KF) were synthesised. The peptides were then separately conjugated to two different sizes (20 nm and 40 nm) of colloidal gold nanoparticles (AuNPs) to study how nanoparticle size is related to conjugate activity. The resulting gold nanoparticle complexes were exhaustively characterised by using different analytical techniques such as UV-vis spectroscopy, amino acid analysis, transmission electron microscopy (TEM), electron energy loss spectroscopy (EELS), and X-ray spectroscopy (XPS).

Furthermore, additional experiments were performed to asses the ability of conjugated nanoparticles to enter HeLa cells and their cellular trajectories. Finally antiproliferation assays were performed to determine their cytotoxic activity against said tumor cell line.

As a general conclusion, we found that the antitumoral activity of Kahalalide F and analogues thereof is enhanced by the conjugation of the compounds with colloidal metal nanoparticles. Thus, in a first aspect, the inventions relates to a colloidal metal nanoparticle conjugated with Kahalalide F, or an analogue thereof.

In the context of the present invention, by the term ‘colloidal metal nanoparticle’ it is understood any water-insoluble metal particle or metallic compound dispersed in liquid water, or forming a hydrosol or a metal sol, having an average size less than 1 μm, i.e. an average size between 1 and 999 nm.

By the term “average size” it is understood the average diameter of the nanoparticle population. The average size of these systems can be measured using standard procedures known by a person skilled in the art, such as differential centrifugal sedimentation, dynamic laser scattering, zeta potential or transmission electron microscopy (TEM). Preferably, the colloidal metal nanoparticles to be used in the present invention have an average particle size ranging from 1 to 500 nm, preferably determined by transmission electron microscopy (TEM). In a preferred embodiment, the average particle size of the colloidal metal nanoparticles is from 5 to 100 nm, more preferably from about 10 to about 60, from about 15 to about 50 and from about 20 nm to about 40 nm and even more preferably from 20 nm to 40 nm. In an even preferred embodiment the average particle size is 20, 21, 22, 23, 24, 25, 26, 27, 28, 29, 30, 31, 32, 33, 34, 35, 36, 37, 38, 39 or 40 nm, and even most preferred is 40 nm.

The metal may be selected from the metals of groups IA, IB, IIB and IIIB of the periodic table, as well as the transition metals, especially those of group VIII. Preferred metals include gold, silver, aluminium, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the Ag¹⁺, Al³⁺, Au³⁺, Ru³⁺, Zn²⁺, Fe³⁺, Ni²⁺, and Ca²⁺ ions. Such metal ions may be present in the complex alone or with other inorganic ions.

A preferred metal is gold, particularly in the form of Au³⁺. An especially preferred form of colloidal gold is HAuCl₄. Colloidal gold nanoparticles are kept in suspension by an inherent negative surface charge that causes the particles to repel one another. In 1857, Michael Faraday manufactured the first nano-sized particles of Au by reducing gold chloride with sodium citrate (Faraday M. Philos. Trans. R. Soc. London, 1857 147, 145-181). Frens (Frens G. Nature Phys. Sci. 1973, 241, 20-22,) and Horisberger (Horisberger M. Biol. Cellulaire, 1979, 36, 253-258,) elaborated on his discovery by demonstrating that the gold to citrate ratio controlled the size of the nanoparticles. Particle size is inversely related to the amount of citrate added to the gold chloride solution: increasing the amount of sodium citrate to a fixed amount of gold chloride results in the formation of smaller particles, while reducing the amount of citrate added to the gold solution results in the formation of relatively larger particles. In a particular embodiment of the invention, the colloidal gold nanoparticles are obtained via the sodium citrate reduction method, see Example 2.

Also in the context of the present invention, by the term ‘conjugated’ it is understood the association between the colloidal metal nanoparticle and Kahalalide F, or an analogue thereof, by means of a direct or indirect bond. This includes covalent and ionic bonds and other weaker or stronger associations that allow for long term or short term association of the Kahalalide compounds with the metal nanoparticle and, optionally, of other additional agents, such as targeting molecules or therapeutic agents.

With the aim of facilitating the binding or association to the Kahalalide F or an analogue thereof, the colloidal metal nanoparticles can be modified by incorporating a reactive group.

In US 2005/0175584, it is described that thiolated alkanes and other thiolated molecules such as polyLys and PEG can act as a bi-functional spacer or cross-linker between the colloidal particle and a therapeutic agent through the thiol.

Thus, methods described for making functionalized colloidal metal nanoparticles comprise the use of reducing agents, wherein a functionalizing polymer containing a free thiol group is added during particle formation. For example, derivatized thiol or derivatized poly-Amino-acid, such as polyethylene glycol (PEG)-thiol or thiolated poly-1-lysine, respectively, are used as reducing agents, thereby incorporating the thiol groups onto the surface of the colloidal metal particles during formation (see for example, US 2005/0175584). Other reducing agents known to those skilled in the art are contemplated to be within the scope of the present invention.

All the above mentioned methods for making functionalised colloidal metal nanoparticles can be used in the present invention for preparing colloidal metal nanoparticles conjugated with Kahalalide F and analogues thereof.

As used herein the terms “functionalized/conjugated colloidal metal particles”, “functionalized nanoparticles”, “conjugated nanoparticles”, “nanoparticle complexes” or the like, are used interchangeably. Similarly, in the present invention, the terms “conjugated”, “functionalized”, “caped” and “coupled” are used as synonyms.

As noted in the introduction, Kahalalide F and analogues thereof have been widely described. They may have the following general formula (I):

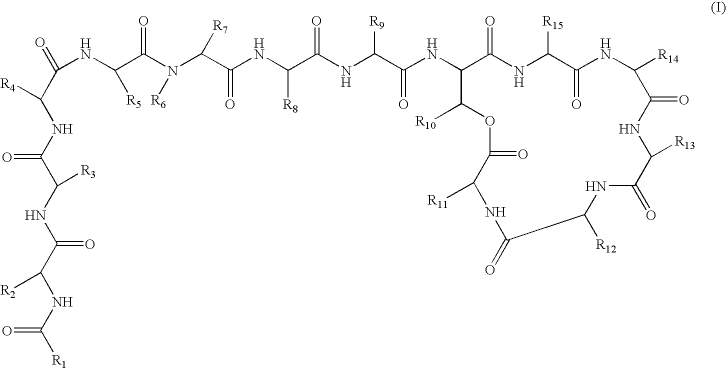

wherein R₁ is selected from hydrogen, substituted or unsubstituted C₁-C₂₅ alkyl, substituted or unsubstituted C₂-C₂₅ alkenyl, and substituted or unsubstituted C₂-C₂₅ alkynyl; and
each R₂, R₃, R₄, R₅, R₆, R₇, R₈, R₉, R₁₀, R₁₁, R₁₂, R₁₃, R₁₄, and R₁₅ are independently selected from hydrogen, substituted or unsubstituted C₁-C₁₂ alkyl, substituted or unsubstituted C₂-C₁₂ alkenyl, substituted or unsubstituted C₂-C₁₂ alkynyl, and substituted or unsubstituted C₁-C₁₂ alkylidene; or R₆ and R₇ together with the corresponding N atom and C atom to which they are attached may form a substituted or unsubstituted heterocyclic group; and pharmaceutically acceptable salts thereof.

In these compounds the groups can be selected in accordance with the following guidance:

Alkyl groups may be branched or unbranched, and preferably have from 1 to about 25 carbon atoms. One more preferred class of alkyl groups has from 1 to about 12 carbon atoms, still more preferably from 1 to about 6 carbon atoms. Even more preferred are alkyl groups having 1, 2, 3 or 4 carbon atoms. Methyl, ethyl, propyl, isopropyl and butyl, including tert-butyl, sec-butyl and isobutyl are particularly preferred alkyl groups in the compounds of the present invention. Another preferred class of alkyl groups has from 5 to about 10 carbon atoms; and even more preferably 6, 7 or 8 carbon atoms. Hexyl, including 4-methylpentyl and 3-methylpentyl, heptyl, and octyl are the most preferred alkyl groups of this class. Yet another preferred class of alkyl groups has from 11 to about 20 carbon atoms; and even more preferably 14, 15 or 16 carbon atoms. Tetradecyl, pentadecyl, and hexadecyl are the most preferred alkyl groups of this class.

Preferred alkenyl and alkynyl groups in the compounds of the present invention may be branched or unbranched, have one or more unsaturated linkages and from 2 to about 25 carbon atoms. One more preferred class of alkenyl and alkynyl groups has from 2 to about 12 carbon atoms, still more preferably from 2 to about 6 carbon atoms. Even more preferred are alkenyl and alkynyl groups having 2, 3 or 4 carbon atoms. Another preferred class of alkenyl and alkynyl groups has from 5 to about 10 carbon atoms; and even more preferably 6, 7 or 8 carbon atoms. Yet another preferred class of alkenyl and alkynyl groups has from 11 to about 20 carbon atoms; and even more preferably 14, 15 or 16 carbon atoms.

Alkylidene groups may be branched or unbranched and preferably have from 1 to 12 carbon atoms. One more preferred class of alkylidene groups has from 1 to about 8 carbon atoms, yet more preferably from 1 to about 6 carbons atoms, and most preferably 1, 2, 3 or 4 carbon atoms. Methylidene, ethylidene and propylidene including isopropylidene are particularly preferred alkylidene groups in the compounds of the present invention.

Suitable aryl groups in the compounds of the present invention include single and multiple ring compounds, including multiple ring compounds that contain separate and/or fused aryl groups. Typical aryl groups contain from 1 to 4 separated or fused rings and from 6 to about 18 carbon ring atoms. Preferably aryl groups contain from 6 to about 10 carbon ring atoms. Specially preferred aryl groups include substituted or unsubstituted phenyl, substituted or unsubstituted naphthyl, substituted or unsubstituted biphenyl, substituted or unsubstituted phenanthryl and substituted or unsubstituted anthryl.

Suitable heterocyclic groups include heteroaromatic and heteroalicyclic groups containing from 1 to 4 separated or fused rings and from 5 to about 18 ring atoms. Preferably heteroaromatic and heteroalicyclic groups contain from 5 to about 10 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl and furopyridyl. Suitable heteroalicyclic groups in the compounds of the present invention contain one, two or three heteroatoms selected from N, O or S atoms and include, e.g., pyrrolidinyl, tetrahydrofuranyl, dihydrofuranyl, tetrahydrothienyl, tetrahydrothiopyranyl, piperidyl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo [3.1.0]hexyl, 3-azabicyclo [4.1.0]heptyl, 3H-indolyl, and quinolizinyl.

The groups above mentioned may be substituted at one or more available positions by one or more suitable groups such as OR′, ═O, SR′, SOR′, SO₂R′, NO₂, NHR′, N(R′)₂, ═N—R′, NHCOR′, N(COR′)₂, NHSO₂R′, NR′C(═NR′)NR′R′, CN, halogen, COR′, COOR′, OCOR′, OCONHR′, OCON(R′)₂, protected OH, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list.

Suitable halogen substituents in the compounds of the present invention include F, Cl, Br and I.

The term “pharmaceutically acceptable salt” refers to any pharmaceutically acceptable salt which, upon administration to the patient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts can be carried out by methods known in the art.

For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts. A preferred salt is trifluoroacetate.

Preferred kahalalide compounds are those of general formula (I) wherein R₁ is a substituted or unsubstituted C₁-C₂₅ alkyl; each R₂, R₃, R₄, R₅, R₈, R₉, R₁₀, R₁₁, R₁₃, R₁₄ and R₁₅ are independently a substituted or unsubstituted C₁-C₁₂ alkyl; R₆ and R₇ together with the corresponding N atom and C atom to which they are attached form a substituted or unsubstituted heterocyclic group; and R₁₂ is a substituted or unsubstituted C₁-C₁₂ alkylidene, or a pharmaceutically acceptable salt thereof.

Even more preferred compounds are those of general formula (I) wherein one or more of the following definitions will apply:

R₁ is 4-methylpentyl or 3-methylpentyl;
R₂ is isopropyl;
R₃ is 1-hydroxyethyl;
R₄ is isopropyl;
R₅ is isopropyl;
R₆ and R₇ together with the corresponding N atom and C atom to which they are attached form a pyrrolidine group;
R₈ is aminopropyl;
R₉ is sec-butyl;
R₁₀ is methyl:
R₁₁ is isopropyl;
R₁₂ is ethylidene;
R₁₃ is benzyl;
R₁₄ is isopropyl; and
R₁₅ is sec-butyl.

Compounds of the following formula (II), and pharmaceutically acceptable salts thereof, are particularly preferred:

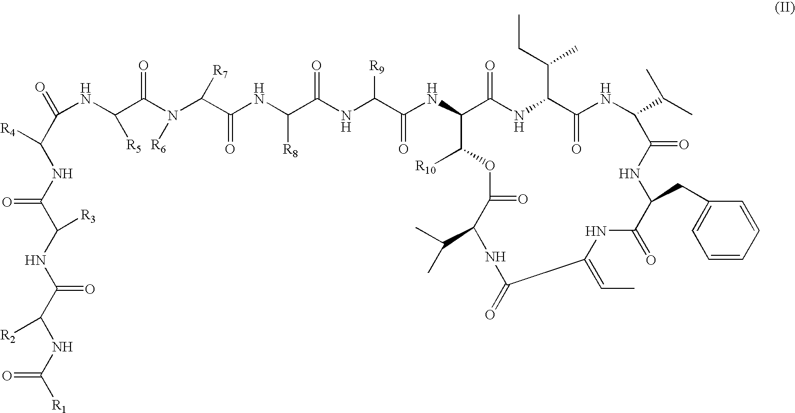

wherein each R₁, R₂, R₃, R₄, R₅, R₆, R₇, R₈, and R₉ have the same meaning given above.

Examples of compounds for the present invention include natural compounds, such as Kahalalide F, and synthetic compounds such as those disclosed in WO 01/58934, WO2005/023846, WO 2004/035613, and Shilabin A G et al. J. Med. Chem. 2007, 50, 4330-4350, which are incorporated herein by reference.

Particularly preferred compounds are those which have been modified in order to incorporate a reactive group that facilitates grafting to the surface of the colloidal metal nanoparticle, for example, carboxyl and/or sulfhydryl groups.

A preferred modification is to incorporate a free sulfhydryl/thiol group that allows the Kahalalide peptide to form a dative bond with the colloidal gold nanoparticles. For example, to replace one or more amino acid residues of the kahalalide structure with cysteine (Cys), in particular to replace one, two, three, four or five valine (Val) residues with cysteine (Cys).

In a particular embodiment Kahalalide F has been modified in the 13th amino acid, valine (D-Val), which has been replaced with cysteine (Cys), in order to allow grafting onto the gold surfaces. In Example 1, the following synthetic epimer analogues of Kahalalide F (P1 and P2) were synthesised:

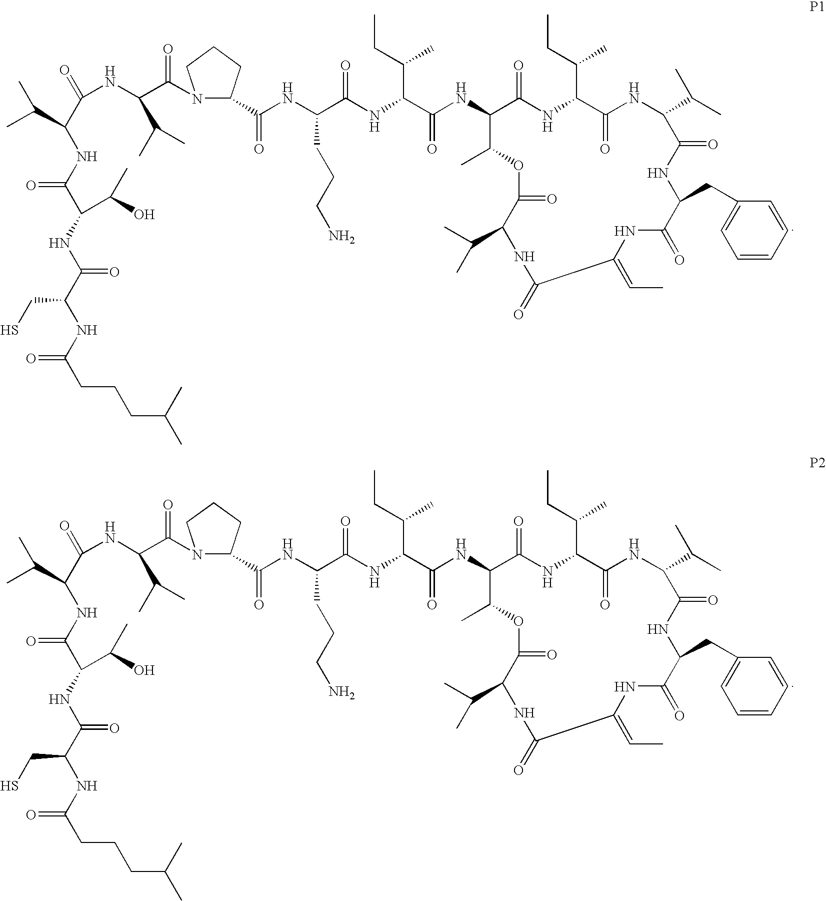

In P1, D-Val¹³ of Kahalalide F was replaced with D-Cys, and in P2, with L-Cys, to determine whether the stereochemistry of the cysteine residue is related to the activity of the gold conjugates. P1 and P2 were synthesised using a previously described Fmoc/tBu solid phase synthesis strategy (Lopez-Macia A et al. J. Am. Chem. Soc. 2001, 123, 11398-11401).

In another aspect, the present invention refers to a Kahalalide F, or an analogue thereof, functionalized colloidal metal nanoparticle which is also conjugated to one or more additional agents, directly or indirectly. The agents may be biologically active agents that can be used in therapeutic applications or detection methods, agents that can be used to alter the biodistribution of the nanoparticle complexes or may be agents that aid in specific targeting of the nanoparticle complexes.

Said agent can be any compound, chemical, therapeutic agent, pharmaceutical agent, drug, biological factor, fragments of biological molecules such as antibodies, proteins, lipids, nucleic acids or carbohydrates; nucleic acids, antibodies, proteins, lipids, nutrients, cofactors, nutriceuticals, anaesthetic, detection agents, an agent that has an effect in the body, an agent that prevents immune detection and/or clearance by the reticuloendothelial system (RES).

Of particular interest are therapeutic agents, as used herein the term “therapeutic agent” refers to any compound or substance having or exhibiting healing powers.

The following are non-limiting examples of some of the agents that can be used in the present invention. One type of agent that can be employed in the present invention includes biological factors including, but not limited to, cytokines, growth factors, fragments of larger molecules that have activity, neurochemicals, and cellular communication molecules. Examples of such agents include, but are not limited to, Interleukin-1 (“IL-1”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin-10 (“IL-10”), Interleukin-11 (“IL-11”), Interleukin-12 (“IL-12”), Interleukin-13 (“IL-13”), Interleukin-15 (“IL-15”), Interleukin-16 (“IL-16”), Interleukin-17 (“IL-17”), Interleukin-18 (“IL-18”), Type I Interferon, Type II Interferon, Tumor Necrosis Factor (“TNFa”), Transforming GrowthFactor-a (“TGF-a”), Lymphotoxin, Migration Inhibition Factor, Granulocyte-Macrophage Colony-Stimulating Factor (“CSF”), Monocyte-Macrophage CSF, Granulocyte CSF, vascular epithelial growth factor (“VEGF”), Angiogenin, transforming growth factor-(“TGF-”), fibroblast growth factor, angiostatin, endostatin, GABA, and acetyl choline.

Another type of agent includes hormones. Examples of such hormones include, but are not limited to, growth hormone, insulin, glucagon, parathyroid hormone, luteinizing hormone, follicle stimulating hormone, luteinizing hormone releasing hormone, estrogen, testosterone, dihydrotestoerone, estradiol, prosterol, progesterone, progestin, estrone, other sex hormones, and derivatives and analogs of hormones.

Yet another type of agent includes pharmaceuticals. Any type of pharmaceutical agent can be employed in the present invention. For example, antiinflammatory agents such as steroids and nonsteroidalantiinflammatory agents, soluble receptors, antibodies, antibiotics, analgesics, angiogenic and antiangiogenic agents, and COX-2 inhibitors, can be employed in the present invention.

Chemotherapeutic agents are of particular interest in the present invention. Non-limiting examples of such agents include DNA-alkylating agents such as cyclophosphamide, ifosfamide, cisplatin, carboplatin, and dacarbazine; antimetabolites such as 5-fluorouracil, capecitabine, 6-mercaptopurine, methotrexate, gemcitabine, cytarabine, and fludarabine; mitotic inhibitors such as paclitaxel, docetaxel, vinblastine, and vincristine; anthracyclines such as daunorubicin, doxorubicin, epirubicin, idarubicin, and mitoxantrone; topoisomerase I and II inhibitors such as topotecan, irinotecan, etoposide, and teniposide; and hormone therapy such as tamoxifen and flutamide.

Immunotherapy agents are also of particular interest in the present invention. Non-limiting examples of immunotherapy agents, include inflammatory agents, biological factors, immune regulatory proteins, and immunotherapy drugs, such as AZT and other derivatized or modified nucleotides.

Another type of agent includes nucleic acid-based materials. Examples of such materials include, but are not limited to, nucleic acids, nucleotides, DNA, RNA, tRNA, mRNA, sense nucleic acids, antisense nucleic acids, ribozymes, DNAzymes, protein/nucleic acid compositions, SNPs, oligonucleotides, vectors, viruses, plasmids, transposons, and other nucleic acid constructs known to those skilled in the art.

Other agents that can be employed in the invention include, but are not limited to, lipid A, phospholipase A2, endotoxins, staphylococcal enterotoxin B and other toxins, heat shock proteins, carbohydrate moieties of blood groups, Rh factors, cell surface receptors, antibodies, cancer cell specific antigens; such as MART, MAGE, BAGE, and HSPs (Heat Shock Proteins), radioactive metals or molecules, detection agents, enzymes and enzyme co-factors.

Of particular interest are detection agents such as dyes or radioactive materials that can be used for visualizing or detecting the sequestered colloidal metal vectors. Fluorescent, chemiluminescent, heat sensitive, opaque, beads, magnetic and vibrational materials are also contemplated for use as detectable agents that are associated or bound to the colloidal metal nanoparticles of the present invention.

Also of particular interest are hydrophilic blockers such as thiol-derivatized polyethylene glycol (PEG-thiol) which may be useful in avoiding detection by the reticuloendothelial system of the nanoparticle complexes and uptake by the liver and spleen.

One or more targeting molecules may be directly or indirectly bound or associated with the colloidal metal. These targeting molecules can be directed to specific cells or cell types, cells derived from a specific embryonic tissue, organs or tissue. Such targeting molecules include any molecules that are capable of selectively binding to specific cells or cell types. In general, such targeting molecules are one member of a binding pair and as such, selectively bind to the other member. Such selectivity may be achieved by binding to structures found naturally on cells, such as receptors found in cellular membranes, nuclear membranes or associated with DNA. The binding pair member may also be introduced synthetically on the cell, cell type, tissue or organ.

Targeting molecules also include receptors or parts of receptors that may bind to molecules found in the cellular membranes or free of cellular membranes, ligands, antibodies, antibody fragments, enzymes, cofactors, substrates, and other binding pair members known to those skilled in the art. Targeting molecules may also be capable of binding to multiple types of binding partners. For example, the targeting molecule may bind to a class or family of receptors or other binding partners. The targeting molecule may also be an enzyme substrate or cofactor capable of binding several enzymes or types of enzymes.

In another aspect, the invention is directed to a colloidal metal nanoparticle conjugated with Kahalalide F or an analogue thereof for use as a medicament. In a preferred aspect, the invention refers to a colloidal metal nanoparticle conjugated with Kahalalide F or an analogue thereof for use as a medicament for treating cancer.

In another aspect, the invention is also directed to the use of a colloidal metal nanoparticle conjugated with Kahalalide F or an analogue thereof, for the manufacture of a medicament for the treatment of cancer.

In yet another aspect, the invention relates to a method of treating cancer comprising administering to a patient in need of such treatment a therapeutically effective amount of a colloidal metal nanoparticle conjugated with Kahalalide F, or an analogue thereof.

Depending on the type of tumor and the development stage of the disease, the treatments of the invention are useful in promoting tumor regression, in stopping tumor growth and/or in preventing metastasis. In particular, the method of the invention is suited for human patients, especially those who are relapsing or refractory to previous chemotherapy. First line therapy is also envisaged.

Preferably, the colloidal metal nanoparticle of the invention is used for the treatment of leukemia, melanoma, breast cancer, colon cancer, colorectal cancer, ovarian cancer, renal cancer, epithelial cancer, pancreatic cancer, lung cancer, cervix cancer, liver cancer, and prostate cancer.

In a further aspect the invention relates to a method for increasing the antitumoral activity of Kahalalide F, or an analogue thereof, which comprises conjugating the Kahalalide F, or the analogue thereof, with a colloidal metal nanoparticle.

In a particular embodiment of the invention, as shown in Example 4, anti-proliferation assays were used to determine the cytotoxic activity of kahalalide-conjugated nanoparticles. The degree of cytotoxic activity of single peptides (P1 and P2), single gold nanoparticle (AuNP) solutions with an average size of 20 nm and 40 nm (AuNP-20 and AuNP-40) and their respective conjugates was determined by the WTS-1 assay in HeLa tumor cells following 24 h of incubation.

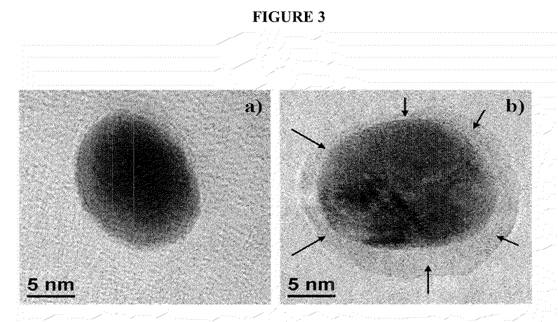

It was surprisingly found that the antitumoral activity of kahalalide-conjugated AuNPs of both sizes (20 nm and 40 nm) was higher than the activity of the respective peptides given alone. Thus, colloidal gold nanoparticles functionalized with Kahalalide F, and analogues thereof, show improved antitumoral activity with respect to the corresponding free Kahalalide F and analogues thereof. While not wishing to be bound by any theory, it is theorized that this increase in the bioactivity of Kahalalide F and its analogues can be the result of the nanoparticle acting as a presenter of the anti-tumour agent, concentrating numerous peptide molecules on its surface. This is in accordance with FIG. 3B and with the results of the quantification of the gold nanoparticle loading, in Example 3.

In addition, nanoparticle size was also observed to be related to in vitro citoxicity. In this regard, AuNP-40 conjugates were slightly more cytotoxic than AuNP-20 conjugates. This effect could be related with a better cell uptake of the AuNP-40 conjugates, as disclosed in Example 4.

In a further aspect, the invention is directed to a pharmaceutical composition comprising a colloidal metal nanoparticle conjugated with Kahalalide F, or an analogue thereof, and a pharmaceutically acceptable vehicle.

The term “vehicle” refers to a diluent, adjuvant, excipient, or carrier with which the conjugated colloidal metal nanoparticles of the invention are administered. The pharmaceutical composition of the invention, if desired, can also comprise, when necessary, additives to enhance, control, or otherwise direct the intended therapeutic effect of the conjugated colloidal metal nanoparticles, and/or auxiliary substances or pharmaceutically acceptable substances, such as pH buffering agents, tensioactives, co-solvents, bulking agents, preservatives, etc. Examples of suitable pharmaceutical vehicles are described in “Remington's Pharmaceutical Sciences” by E. W. Martin. Additional information about said vehicles can be found in any handbook of Pharmaceutical Technology (i.e., galenic pharmacy).

The pharmaceutical composition of the invention will be formulated according to the chosen route of administration. The pharmaceutical composition of the invention can be administrated by any suitable route, including but not limited to oral, rectal, transdermal, ophthalmic, nasal, topical, vaginal or parenteral. In a particular embodiment, the pharmaceutical composition is formulated in order to be suitable for parenteral administration to a subject, e.g., a human being, preferably by intravenous, intramuscular, intraperitoneal or subcutaneous administration. Illustrative, non limiting examples of suitable formulations for parenteral administration are solutions, suspensions, emulsions, lyophilized compositions and the like. The administration of the pharmaceutical composition of the invention to the subject in need thereof can be carried out by conventional means.

As used herein, the term “subject” refers to an animal, preferably a mammal including a non-primate (e.g., a cow, pig, horse, cat, dog, rat, or mouse) and a primate (e.g., a monkey, or a human). In a preferred embodiment, the subject is a human.

In a particular embodiment, the administration of the pharmaceutical composition of the invention will be by intravenous route of administration and will include an intravenous delivery through standard devices, e.g., a standard peripheral intravenous catheter, a central venous catheter, or a pulmonary artery catheter, etc. In any case, the pharmaceutical composition of the invention will be administrated using the appropriate equipments, apparatus, and devices which are known by the skilled person in art.

The dosage and schedule of administration of the pharmaceutical composition of the invention will vary according to the particular formulation, the mode of administration, and the particular situs and tumour being treated. Other factors like age, body weight, sex, diet, rate of excretion, condition of the subject, drug combinations, reaction sensitivities and severity of the disease shall be taken into account. Administration can be carried out continuously or periodically within the maximum tolerated dose.

In a preferred embodiment, the pharmaceutical composition is formulated in order to be suitable for intravenous administration. Preferred infusion times are of up to 24 hours, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks.

In a further aspect of the invention, the conjugated colloidal metal nanoparticles and the pharmaceutical compositions of the invention can be used with other drugs to provide a combination therapy for the treatment of cancer. The other drugs may form part of the same composition, or be provided as a separate pharmaceutical composition for administration at the same time or a different time. The identity of the other drug is not particularly limited, and suitable candidates include: DNA-alkylating drugs (such as cyclophosphamide, ifosfamide, cisplatin, carboplatin, dacarbazine), antimetabolites (such as 5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase I and II inhibitors (such as topotecan, irinotecan, etoposide, teniposide), and hormone therapy (such as tamoxifen, flutamide).

In a particular embodiment, said additional drug is administered simultaneously or sequentially to the conjugated colloidal metal nanoparticles of the present invention, spaced out in time, in any order, i.e. first the conjugated colloidal metal nanoparticles of the invention, then the additional drug can be administered, or first the additional drug and then the conjugated colloidal metal nanoparticles of the invention can be administered. In another alternative embodiment the conjugated colloidal metal nanoparticles of the invention and an additional drug are simultaneously administered.

In a particular embodiment of the invention, as shown in Example 5, conjugated colloidal gold nanoparticles of both sizes (20 and 40 nm) were found in the lysosome-like structures in much higher quantities than those unconjugated. This may be due to the fact that the Kahalalide peptides guide the conjugated nanoparticles intracellularly to the lysosomal compartment.

A further aspect of the invention is directed to a colloidal metal nanoparticle conjugated with kahalalide F, or an analogue thereof, which is further conjugated to an additional agent and its use for intracellularly delivering said additional agent to lysosome-like compartments.

In a preferred embodiment, said additional agent is a therapeutic agent. The term “therapeutic agent” has been previously described.

Accordingly, in another aspect the present invention provides a method for selectively delivering a therapeutic agent to subcellular targets, in particular to lysosome-like compartments, which comprises the conjugation of said therapeutic agent with a kahalalide conjugated nanoparticle of the invention.

In another aspect, the present invention relates to a method for obtaining a colloidal metal nanoparticle conjugated with Kahalalide F, or an analogue thereof, comprising the following steps:
(i) obtaining colloidal metal nanoparticles by reduction of a solution of a metal salt;
(ii) mixing a solution of Kahalalide F or an analogue thereof with the colloidal metal nanoparticle solution obtained in step i) for a sufficient period of time to form conjugated nanoparticles, wherein the Kahalalide F, or an analogue thereof, is in excess with respect to the colloidal metal nanoparticle;
(iii) optionally, admixing the conjugated nanoparticles with an additional agent to form a reaction mixture and incubating the reaction mixture for a sufficient period of time to allow the conjugated nanoparticles to bind said additional agent; and
(iv) isolating the conjugated colloidal metal nanoparticles.

Isolation of the conjugated colloidal metal nanoparticles can be performed by techniques, generally known by a person skilled in the art, such as filtration, dialysis, centrifuge methods, affinity columns, magnetic separation, methods of precipitation using organic solvents such as methanol, ethanol, etc. Preferably, the isolation of the functionalized colloidal metal nanoparticles of the present invention is performed by dialysis.

The amount of conjugated peptide and optionally, a further agent, bound to the surface of the colloidal metal nanoparticle can be determined by quantitative methods for determining proteins, therapeutic agents or detection agents, such as ELISA or spectrophotometric methods.

The present invention will be further illustrated with reference to the following Examples which aid in the understanding, but are not to be construed as limitations thereof.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### Example 1

### Synthesis of Kahalalide F analogues

Both peptides (P1 and P2) were synthesised using a previously described Fmoc/tBu solid-phase synthesis strategy (Lopez-Macia, A et al.; J. Am. Chem. Soc. 2001, 123, 11398).

### Material and Reactives

Cl-TrtCl-resin (100 mg, 1.56 mmol/g) and protected Fmoc-L-amino acids and Fmoc-D-amino acids derivatives were purchased from Iris Biotech GmbH (Marktredwitz, Germany), Luxembourg Industries (Tel-Aviv, Israel), Neosystem (Strasbourg, France), Calbiochem-Novabiochem AG (Laüfelfingen, Switzerland) and Bachem AG (Bubendorf, Switzerland). Diisopropylcarbodiimide (DIC) was obtained from Fluka Chemika (Buchs, Switzerland), HOAt from GL Biochem (Shanghai, China), PyBOP from Calbiochem-Novabiochem AG and N,N-diisopropylethylamine (DIEA) from Albatros Chem. Inc. (Montreal, Canada). Solvents for peptide synthesis and RP-HPLC equipment were obtained from Scharlau (Barcelona, Spain). Trifluoroacetic (TFA) acid was supplied by KaliChemie (Bad Wimpfen, Germany). Other chemicals used were obtained from Aldrich (Milwaukee, Wis., USA) and were of the highest purity commercially available.

HPLC was performed using a Waters Alliance 2695 (Waters, Mass., USA) chromatography system with a PDA 995 detector, a reverse-phase Symmetry C18 (4.6×150 mm) 5-μm column and linear gradient MeCN with 0.036% TFA into H₂O with 0.045% TFA. The system was run at a flow rate of 1.0 mL/min. HPLC-MS was performed using a Waters Alliance 2796 with a UV/Vis detector 2487 and ESI-MS Micromass ZQ (Waters) chromatography system, a reversed-phase Symmetry 300 C18 (3.9×150 mm) 5-μm column, and H₂O with 0.1% formic acid and MeCN with 0.07% formic acid as mobile phases. Mass spectra were recorded on a MALDI Voyager DE RP time-of-flight (TOF) spectrometer (PE Biosystems, Foster City, Calif., USA).

### Solid-Phase Synthesis

The two peptides were synthesized using the Fmoc solid-phase strategy in polypropylene syringes fitted with polyethylene porous disks. Side chains of Fmoc aminoacids were protected as follows: Thr was protected with the tert-butyl group (tBu) and Cys with the trityl group (Trt). Solvents and soluble reagents were removed by suction. Washings between deprotection, couplings and subsequent deprotection steps were carried out with DMF and DCM using 10 mL of solvent/g of resin each time. The Fmoc group was removed by treatment with piperidine:DMF (1:4) for 20 min. All syntheses were performed on Cl-TrtCl resin (100 mg each peptide). Couplings of all Fmoc-aa-OH (4 equiv) except with the dipeptide Alloc-Phe-(Z)Dhb-OH were performed with DIC (4 equiv) and HOAt (4 equiv) in DMF for 1.5 h at room temperature. Recouplings were done with PyBOP (4 equiv), HOAt (4 equiv) and DIEA (12 equiv) for 1.5 h at room temperature. The coupling of dipeptide Alloc-Phe-(Z)Dhb-OH (1 equiv) was performed with DIEA (1 equiv) for 5 min, DIEA (2 equiv) for 1 h. The resin was washed with DMF and DCM after each coupling. Couplings were monitored using Kaiser (Kaiser, E. C., R. L. Bossinger, C. D. Cook, P. I., Anal. Biochem., 1969. 34, 595) or de Clercq (Madder, A. F., N. Hosten, NGC. De Muynck, H. De Clercq, P. J. Barry, J. Davis, A. P., Eur. J. Org. Chem., 1999, 2787) methods. After each coupling, the capping steps were performed with Ac₂O:DIEA:DMF (4:2:94) but with the dipeptide Alloc-Phe-(Z)Dhb-OH the capping step was done with Methanol (300 μL). Alloc group was removed with Pd(PPh₃)₄ (0.1 equiv) in the presence of PhSiH₃ (10 equiv) during 15 min and it was repeated three times. To cleave the peptide from the solid support, the resin was washed with DCM (3×1 min), dried, and then washed again with a mixture TFA:DCM (1:99) (6×1 min) and washed with DCM and the filtered was collected in a round-bottom flask which contained 100 μL H₂O and 50 μL DIEA. TFA was then removed by evaporation under reduced pressure, and peptides were precipitated with cold anhydrous TBME, dissolved in H₂O:MeCN (1:1) and then lyophilized. The two peptides were cycled upon dissolution in a PyAOP (4 equiv) and DIEA (8 equiv) solution. It was then stirred at room temperature for 24 h (Ellman G. L., Arch. Biochem. Biophys., 1959. 82,70). Cyclization was easily monitored either by Ellman's test and RP-HPLC. The solution was then evaporated with N₂, dissolved in H₂O:MeCN (1:1) and then lyophilized. The crude peptides were purified by semi-preparative HPLC.

### Example 2

### Synthesis and Characterization of Gold Nanoparticles

Gold nanoparticles were produced by reduction of hydrogen tetrachloroaurate (HAuC14×H₂0; Aldrich, Milwaukee, Wis., USA).

For the synthesis of gold nanoparticles having a size of 20 nm, HAuC14×H₂O (8.7 mg) was dissolved in water (1 mL), and the tetrachloroaurate solution was added to a sodium citrate solution (100 mL, 2.2 mM in water) at 150° C. reflux and the reaction was allowed to continue under uniform and vigorous stirring until a red wine colour was observed, following the protocol described by Sagara T et al. J. Phys. Chem. B 2002, 106, 1205-1212.

On the other hand, for the synthesis of gold nanoparticles having a size of 40 nm, it was used the same procedure as above except in that a minor amount of reducing agent was used (100 mL of a 1.22 mM solution of sodium citrate in water).

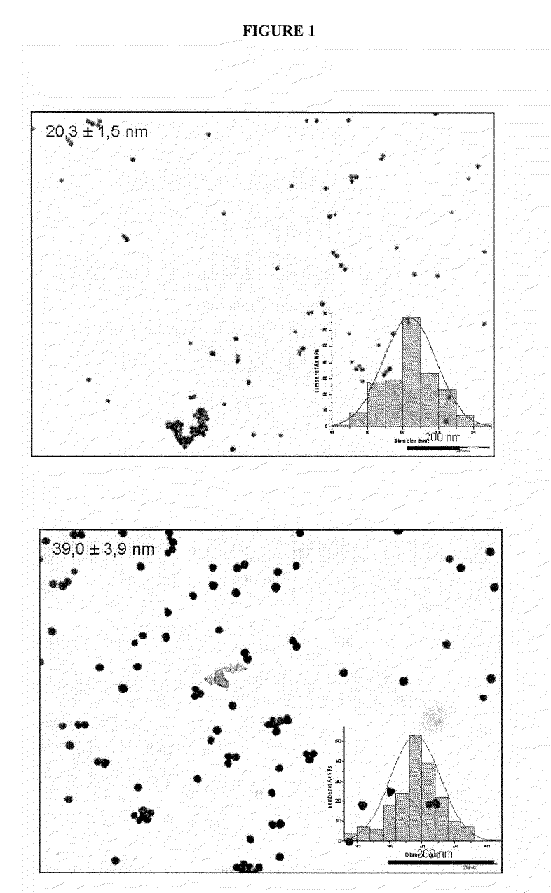

Unconjugated gold nanoparticles were characterised using Transmission electron microscopy (TEM). Accordingly, drops of bare gold nanoparticles were deposited over carbon-coated Formvar films on copper grids. The samples were viewed with a transmission electron microscope (JEOL JEM 1010 (Japan)) at an accelerating voltage of 80 kV. The images shown in FIGS. 1A and 1B were obtained with a CCD Megaview III (SIS) camera (Münster, Germany).

It was observed that the gold nanoparticles were free from aggregation, and homogenous in size with a size variation of 7% for the nm nanoparticles (FIG. 1A) and 10% for the 40 nm nanoparticles (FIG. 1B).

### Example 3

### Preparation and Characterisation of Conjugated Gold Nanoparticles

The peptides (P1 and P2), obtained in example 1, were separately conjugated with the two types of gold nanoparticles (20 nm and 40 nm), obtained in Example 2, in order to study how nanoparticle size is related to conjugate activity.

An excess of peptide was used for conjugation (Kogan M J et al. Nano Lett. 2006, 6(1), 110-115). Peptide solutions (1 mg of peptide dissolved in 1 mL water) were added dropwise to 10 mL of a gold nanoparticle solution (solution of 2.2 mM sodium citrate in water) at room temperature with magnetic stirring. Agitation was then maintained for 15 min. The gold complexes were then purified by dialysis over three days in a Spectra/membrane (MWCO: 6-8000) against 2.2 mM sodium citrate. The solution was changed six times in order to eliminate the excess peptide (P1 or P2).

The gold nanoparticle conjugates were exhaustively characterised using UV-vis spectroscopy, amino acid analysis, transmission electron microscopy (TEM), electron energy loss spectroscopy (EELS) and X-ray spectroscopy (XPS).

### UV-Vis Spectroscopy

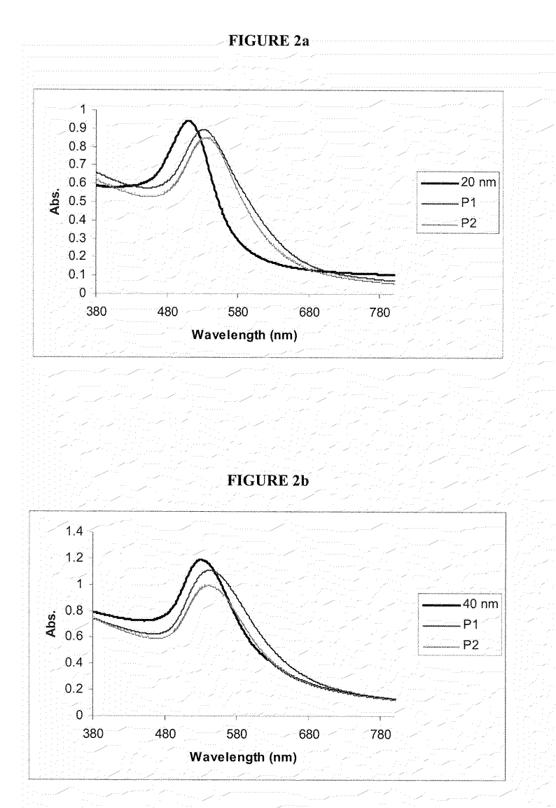

UV-vis absorption spectra of each size of gold nanoparticles were recorded at room temperature with a 2501PC UV-vis recording spectrophotometer (Shimadzu Corporation, Kyoto, Japan). A characteristic shift in the surface plasmon resonance band (520 nm for the 20 nm size nanoparticle, and 530 nm for the 40 nm size nanoparticle) revealed a change in the AuNP surface. All the gold colloids displayed a single absorption peak in the visible range between 510 and 550 nm. The wavelength of the maximum absorption was longer for the 40 nm-sized conjugates than for the 20 nm-sized conjugates (FIG. 2).

### High Resolution Transmission Electron Microscopy (HRTEM)

Drops of gold nanoparticles conjugated to both P1 and P2 under uranyl acetate staining were deposited over carbon-coated Formvar films on copper grids. To obtain the transmission electron microscopy (HRTEM) results, a field emission gun microscope JEOL 2010F was used, working at 200 kV and with a point-to-point resolution of 0.19 nm.

FIG. 3 shows the high-resolution TEM micrographs (HRTEM) of 20 nm gold nanoparticles when uncoated (FIG. 3A) and when coated with P1 (FIG. 3B). The presence of a layer around the nanoparticle core (FIG. 3B) corresponding to the peptide was observed upon uranyl acetate staining. As observed, the peptide covered the whole surface of the nanoparticle and increased the hydrodynamic size of the peptide-capped nanoparticle.

In addition, EELS (electron energy loss spectroscopy) and XPS (X-ray photoelectron spectroscopy) were used to confirm the presence of S—Au bonds on the surface.

### Electron Energy Loss Spectroscopy (EELS)

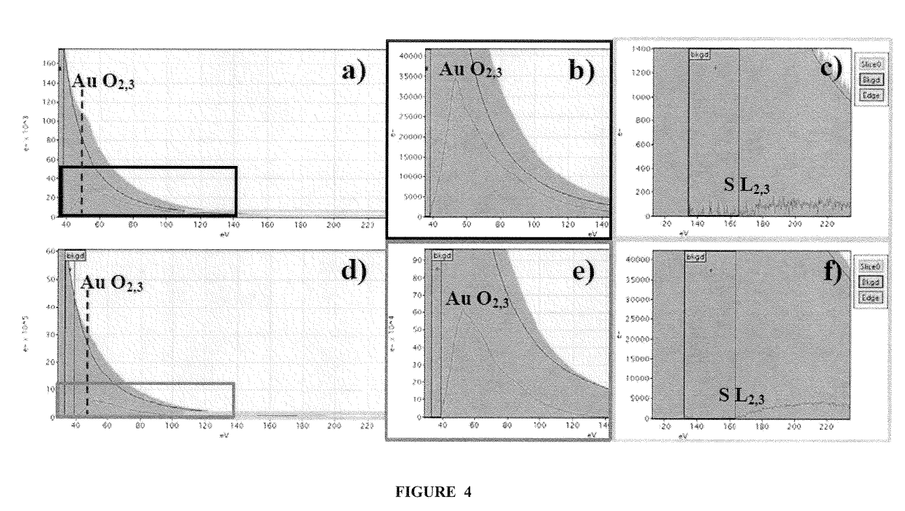

Electron energy loss spectra (EELS) shown in FIG. 4 were obtained in a Gatan Image Filter (GIF 2000) coupled to the JEOL 2010F microscope, with an energy resolution of 1.2 eV.

An accurate analysis of the electron energy loss near edge spectra (ELNES) at the Au O_{2,3} edge (54 eV), showed that the edge shape slightly changed when obtaining the spectrum on the unconjugated Au nanoparticles with regard to the P1-conjugates (FIGS. 4B and 4E, respectively). The change of the ELNES shape at the Au O_{2,3} edge, can be directly attributed to a bonding variation on some of the Au surface atoms.

To determine the cause of this bonding variation we also analyzed the S L_{2,3} edge placed at around 165 eV. In this case, energy filtered spectra obtained on the Au conjugated nanoparticles surface showed a clear signal at around 165 eV (FIG. 4F), indicating the presence of S atoms on the Au nanoparticle surface. The same energy region analysed on the unconjugated nanoparticles surface showed only a noise signal, as indicated in FIG. 4C. As the unconjugated sample was assumed to have a low quantity of S atoms, it was used to evaluate the signal-to-noise ratio at the S L_{2,3} energy region. The average ratio between the maximum on the Au O_{2,3} peak, and the maximum noise signal after background subtraction found at the S L_{2,3} energy region, was 4·10⁻³ for the unconjugated nanoparticles. However, values of up to 8·10⁻³ were found for the functionalised nanoparticles, indicating that the increase in the signal to noise ratio may be due to the bound S atoms. This last result corroborates the presence of S atoms on the Au surface, which would be responsible for the slight change in the Au O_{2,3} ELNES spectra due to their bonding. Consequently, the presence of bound S atoms would indicate the functionalisation of the Au nanoparticles with the P1 peptides.

### X-Ray Spectroscopy (XPS)

The gold colloids were further characterised by X-ray photoelectron spectroscopy (XPS). The XPS studies were carried out on P1-conjugate and unconjugated gold nanoparticles deposited on poly(methylmeta)acrylate surfaces (PMMA). This polymeric surface was used to minimise interference coming from the substrate, a problem commonly observed when sulphur-containing compounds are analysed on silicon surfaces. Usually XPS characterisations are performed on silicon oxide surfaces. Silicon surfaces present two signals corresponding to Si2s and Si2p at 165 and 167 eV, respectively. The Si2s and S2 signals overlapped. The use of PMMA instead of a silicon surface was in order to avoid interference. XPS characterisation of the polymer was performed to discard sulphur impurities.

XPS studies were performed by depositing a drop of gold nanoparticles over PMMA surfaces (GoodFellow; Huntingdon, United Kingdom) and then drying the samples under reduced pressure before analysis. P1 functionalised gold surfaces were obtained by immersion of gold surfaces (Arrandee; Germany) and P1 (0.1 mg) in a CHCl₃ (1 mL) solution for 24 h.

The XPS spectra was centred at 163.2 eV. Based on the various chemical environments of the sulphur atom, two different groups of chemical states can be differentiated. One group corresponds to the sulphur present in the unreacted peptide and the second group corresponds to the chemisorbed sulphur. One can distinguish subgroups, such as the difference in chemical shift induced by various metal adsorbing sites (Bensebaa F. Surface Science, 1998, 405, L472-L476).

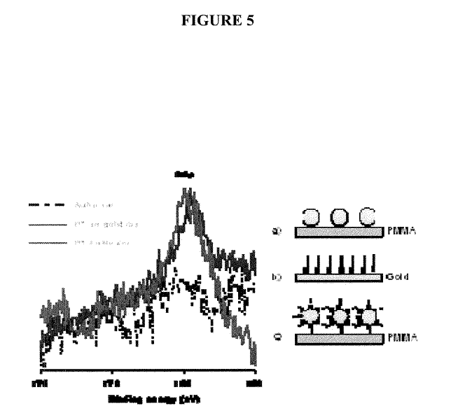

The S₂ₚ spectrum, shown in FIG. 5, gave a weak signal due to the presence of only one sulphur atom per attached peptide. The signal consists of a broad band with a maximum at 163.2 eV that corresponds to sulphur grafted onto gold. Although S_{2p3/2} and S_{2p1/2} signals can usually be observed separately, we observed a single, broad band, presumably due to shielding of the electron emission by the large peptide. A similar S₂ₚ signal was obtained when the peptide was on a non-functionalised gold surface (Barr TL. Modern ESCA: the principles and practice of X-ray photoelectron Spectroscopy. CRC Press, Boca raton, FL, 1994).

### Quantification of Gold Nanoparticle Loading

To determine the degree of conjugation of the peptide with the AuNP, non-dialysed aliquots of the conjugated solutions (2.5 mL) were centrifuged at 13,500 rpm for 30 min. The supernatant was lyophilised, and then analysed by HPLC to determine the amount of unconjugated peptide. Approximately 85% of the peptide used in the conjugation was thus determined to be complexed to the gold nanoparticles. The number of peptides per particle was calculated by dividing the concentration of grafted peptide by the amount of gold nanoparticles in solution, which was determined spectrophotometrically. The molar extinction coefficients of the gold colloids were obtained from the literature (Jain, P. et al., J. Phys. Chem. B 110, 7238-7248), showing ratios of 73,500 peptides per 20 nm particle, and 58,800 peptides per 40 nm particle. However, assuming that the surface of a 20 nm AuNP is 1,250 nm², and that the surface of a molecule in an extended conformation is 0.6 nm², then the theoretical number of molecules that would completely cover a 20 nm AuNP surface is only 2,090. We therefore assumed that the nanoparticles were capped with a multilayer formed by self-assembled peptide molecules.

### Amino Acid Analysis

The integrity of the peptides on the gold surface was confirmed by amino acid analysis.

Amino acid analysis was carried out by the AccQ.Tag method after acid hydrolysis with HCl (6N) for 24 hours at 110° C. The analysis was performed in a Waters Delta 600 RP-LC system with UV detection at 254 nm.

The relationship among the concentrations of the amino acids valine, isoleucine and proline is 4:2:1 in both P1 and P2. The same relationship was observed on the gold conjugates by amino acid analysis (Table I), demonstrating that the peptides maintained structural integrity during conjugation and dialysis.

TABLE I

| | Val (mM) | Ile (mM) | Pro (mM) |
|---|---|---|---|
| | (4) | (2) | (1) |
| P1 20 nm gold nanoparticle | 0.2486 | 0.1279 | 0.0663 |
| P2 20 nm gold nanoparticles | 0.9465 | 0.4717 | 0.2925 |
| P1 40 nm gold nanoparticle | 0.4512 | 0.2299 | 0.1326 |
| P2 40 nm gold nanoparticle | 1.1504 | 0.7323 | 0.4902 |

### Example 4

### Antiproliferation Assay

The degree of cytotoxic activity of single peptides (P1 and P2), single gold nanoparticle solutions (AuNP-20 and AuNP-40) and their respective conjugates was determined by cell viability testing using the WST-1 assay in human cervical epithelium HeLa tumor cells, following 24 h of incubation. Each assay was run in sextuplicate, and the whole experiment was run in triplicate.

HeLa cell line (ATTC no CCL-2) was maintained in Dulbecco Modified Eagle's Minimal Essential Medium (DMEM) low glucose medium (Biological Industries) containing 10% foetal calf serum (FCS), 2 mM glutamine, 50 U/mL penicillin, and 0.05 g/mL streptomycin at 37° C. in a controlled 5% CO₂ atmosphere.

For cell viability experiments, exponentially growing HeLa cells were detached from the culture flasks using a trypsin-0.25% ethylenediaminetetraacetic acid (EDTA) solution and the cell suspension was seeded at a concentration of 3.5×10³ cells/cm² onto glass coverslips (Nalge Nunc International, Rochester, N.Y.). WST 1 assays were carried out 24 h later, when the confluence was approximately 70-80%. Non-adherent cells were washed away and attached cells were incubated at 37° C. in 5% CO₂ in DMEM with a known concentration of gold nanoparticles.

WST-1 Assay.

For each assay, 3.5×10³ cells/cm² were seeded onto a 96-well plate (Nalge Nunc) and cultured for 24 h. The conjugates were added at a peptide concentration of 1×10⁻⁵ M, assuming that the 85% of the initial amount of peptides was in the gold nanoparticle solution either grafted onto the gold surfaces or formed a multi-layer around them. Cells were incubated for 24 h at 37° C. under a 5% CO₂ atmosphere. After 20 h, 10 μL of WST 1 were added. The cells with the conjugate solutions were incubated for a further 4 h.

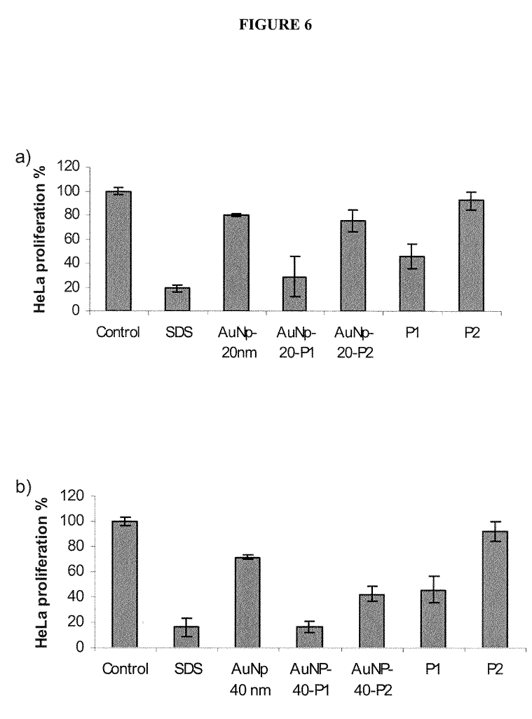

The results showed that both gold unconjugated nanoparticle solutions displayed residual cytotoxicity against HeLa cells, determined in 20% inhibition for AuNP-20 and 30% inhibition for AuNP-40 (FIG. 6). Regarding the cytotoxicity of the single peptides, P2 resulted to be less cytotoxic than P1 (10 vs 50% inhibition, respectively). Additive cytotoxicity was found for P1 conjugates (both, AuNP-20-P1 and AuNP-40-P1) as well as for AuNP-20-P2. However, AuNP-40-P2 resulted in a more than additive cytotoxicity (60% inhibition) compared to the corresponding single components. This could be the result of the better cell uptake of the former, which would be consistent with the findings of Chithrani et al. (Nano Letters, 2007, 7, 1542-1550). The authors reported that among nanoparticles of different sizes, those of 50 nm demonstrated the highest level of uptake by HeLa cells. In agreement with these results, Osaki et al. (J. Am. Chem. Soc. 2004, 126, 6520-6521) qualitatively showed that 50 nm nanoparticles entered the cells via receptor-mediated endocytosis more efficiently than smaller ones.

On the other hand, both 20 and 40 nm P2 gold conjugates showed less cytotoxicity than the P1-conjugates. These results are in accordance with the cytotoxic activity observed for the unconjugated peptides.

Thus, we report that the antitumoral activity of Kahalalide F and analogues thereof can be increased by conjugating these compounds with colloidal metal nanoparticles.

### Example 5

### Intracellular Nanoparticle Localisation

### Confocal Laser Scanning Microscopy (CLSM).

In order to study the influence of the conjugated peptides on the penetration and distribution of the gold nanoparticles in cells, both conjugated and unconjugated nanoparticles were studied by confocal microscopy by observation of their reflections. The cells were fixed with paraformaldehyde, and then the membranes and nuclei were stained.

HeLa cells were plated at a concentration of 2.5×10³ cells/cm² on glass coverslips, grown to 60% confluence and then incubated at 37° C. under a 5% CO₂ atmosphere with either P1- and P2-nanoparticle complexes. The conjugates were added at a peptide concentration of 1×10⁻⁵ M. After 24 hours, the coverslips were rinsed extensively with phosphate-buffered saline (PBS), and the cells were fixed with 4% paraformaldehyde in PBS for 20 minutes at room temperature and then rehydrated in PBS. Once the cells were fixed, the coverslips with cells were mounted onto glass slides with Mowiol mounting media (Calbiochem, Calif.), and then allowed to dry overnight prior to microscopy analysis. The samples were examined using an Olympus Fluoview 500 confocal microscope with a 60×/1.4 NA objective.

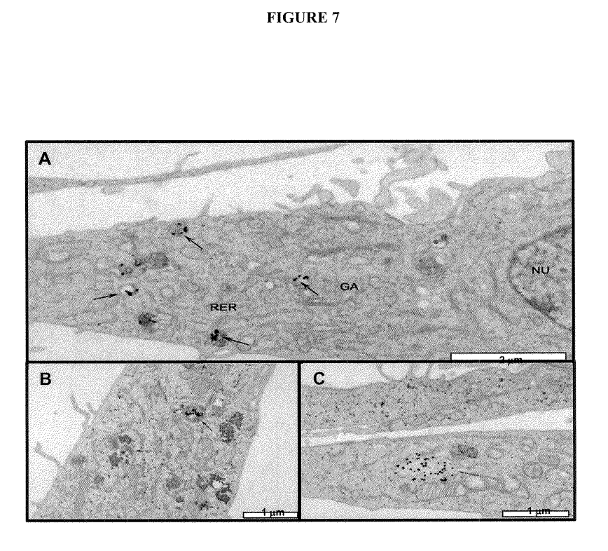

FIG. 7 shows that there are substantial differences between the unconjugated and conjugated gold nanoparticles. Moreover, there are differences between the 20 nm and 40 nm conjugates. Whilst both the conjugated and unconjugated nanoparticles entered the cytoplasm, their fate once inside HeLa cells differed. The unconjugated AuNPs were found in different lysosomes-like bodies throughout the cytoplasm, but only in small quantities. In contrast, the conjugated nanoparticles were primarily found in lysosomes-like compartments that are very close to the nuclear region.

### Transmission Electron Microscopy (TEM)

Cellular localisation of the gold-conjugates was also studied by TEM.

HeLa cells were incubated for 24 h with either conjugated or unconjugated gold nanoparticles. Cells were fixed with 2.5% glutaraldehyde in phosphate buffer, and then kept in the fixative at 4° C. for 24 h. The cells were then washed with the same buffer, and post-fixed with 1% osmium tetraoxide in the same buffer containing 0.8% potassium ferricyanide at 4° C. The samples were then dehydrated in acetone, infiltrated with Epon resin for 2 days, embedded in the resin, and polymerised at 60° C. for 48 hours. Ultrathin sections were obtained using a Leica Ultracut UCT ultramicrotome, and then mounted on Formvar-coated copper grids. The sections were stained with 2% uranyl acetate in water and lead citrate, and then observed under a JEM-1010 electron microscope (Jeol, Japan).

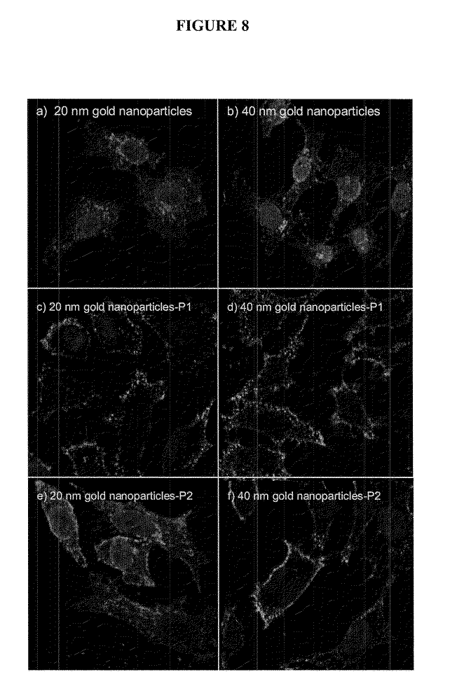

TEM images show that gold particles were localized in lysosomes-like structures (FIG. 8). There was no difference in localisation between the 20 nm and 40 nm particles, whether in the case of the unconjugated or conjugated AuNPs in incubated HeLa cells. However, a substantial difference between conjugated and free AuNPs was observed: both sizes of conjugated AuNPs were found in the lysosome-like structures in much higher quantities than were the unconjugated AuNPs. This may be due to the fact that it is the peptide which directs the AuNPs to the lysosome-like structures. The unconjugated AuNPs were found in different lysosome-like bodies throughout the cytoplasm, but only in small quantities. In contrast, the conjugated nanoparticles were primarily found in lysosome-like compartments that are very close to the nuclear region.

In conclusion, substantial differences in localisation between bare and conjugated AuNPs were found. However, despite the higher anti-proliferative activity of the 40 nm conjugated AuNPs compared to the 20 nm ones, no difference in cellular localisation was observed due to the particle size.

1. A colloidal metal nanoparticle conjugated with Kahalalide F or an analogue thereof.

2. The conjugated nanoparticle according to claim 1, wherein the Kahalalide F analogue is conjugated to the colloidal metal nanoparticle by means of a free thiol group.

3. The conjugated nanoparticle according to any of claim 1 or 2, wherein the colloidal metal is gold.

4. The conjugated nanoparticle according to any of the preceding claims, wherein the nanoparticle has an average particle size ranging from 1 nm to 500 nm.

5. The conjugated nanoparticle according to claim 4, wherein the nanoparticle has an average particle size ranging from 5 nm to 100 nm.

6. The conjugated nanoparticle according to claim 4, wherein the nanoparticle has an average particle size ranging from 20 nm to 40 nm.

7. The conjugated nanoparticle according to any of the preceding claims, wherein the nanoparticle is further conjugated to an additional agent.

8. The conjugated nanoparticle according to claim 7, wherein said additional agent is a therapeutic agent.

9. (canceled)

10. (canceled)

11. (canceled)

12. A pharmaceutical composition comprising a conjugated nanoparticle, as defined in any of claims 1 to 8, and a pharmaceutically acceptable vehicle.

13. (canceled)

14. A method for increasing the antitumoral activity of Kahalalide F, or an analogue thereof, which comprises conjugating the Kahalalide F, or an analogue thereof, with a colloidal metal nanoparticle to obtain a colloidal metal nanoparticle as defined in any of claims 1 to 6.

15. (canceled)

16. (canceled)

17. (canceled)

18. (canceled)

19. (canceled)

20. (canceled)

21. (canceled)

22. A method for obtaining a conjugated nanoparticle as defined in any of claims 1 to 8, comprising the following steps:
(i) obtaining colloidal metal nanoparticles by reduction of a solution of a metal salt;
(ii) mixing a solution of Kahalalide F or an analogue thereof with the colloidal metal nanoparticle solution obtained in step i) for a sufficient period of time to form conjugated nanoparticles, wherein the Kahalalide F, or an analogue thereof, is in excess with respect to the colloidal metal nanoparticle;
(iii) optionally, admixing the conjugated nanoparticles obtained in step ii) with an additional agent to form a reaction mixture and incubating the reaction mixture for a sufficient period of time to allow the conjugated nanoparticles to bind said additional agent; and
(iv) isolating the conjugated colloidal metal nanoparticles.

23. The method according to claim 22, wherein the kahalalide F analogue comprises a free thiol group.

24. The method according to any of claim 22 or 23, wherein the colloidal metal is gold.

25. The method according to any of claims 22 to 24, wherein the nanoparticle has an average particle size ranging from 1 nm to 500 nm.

26. The method according to claim 25, wherein the nanoparticle has an average particle size ranging from 5 nm to 100 nm.

27. The method according to claim 25, wherein the nanoparticle has an average particle size ranging from 20 nm to 40 nm.

28. A method of treating cancer comprising administering to a patient in need of such treatment a therapeutically effective amount of a conjugated nanoparticle as defined in any of claims 1 to 8.
